(19) United States
(12) Patent Application Publication
(10) Pub. No.: US 2002/0061308 A1
(43) Pub. Date: May 23, 2002

(54) Bcl-2 gene proteins conjugated to a ligand for a cell surface antigen of a specific cell type

(76) Inventor: Nancy T. Chang, Houston, TX (US)

Correspondence Address: ERIC P. MIRABEL, ESQ., WINSTEAD SECHREST & MINICK P.C., 2400 BANK ONE CENTER, 910 TRAVIS STREET, HOUSTON, TX, 77002, US

(21) Appl. No.: 09/974,338

(22) Filed: Oct. 10, 2001

Related U.S. Application Data
(60) Provisional application No. 60/238,942, filed on Oct. 10, 2000.

(51) Int. Cl. A61K 39/395; C07K 16/46

(52) U.S. Cl. 424/178.1; 530/391.1

(57) ABSTRACT

The invention is related to conjugates comprised of a pro-apoptotic protein encoded by Bak, Bak-2 or Bax genes and a ligand specifically targeting a cell surface antigen. The cell surface antigen is expressed on tumor cells, cells infected with bacteria or virus, or cells of specific functions, but it is not expressed or expressed only to a limited extent on other cell types. The ligand my be an organic molecule, an oligonucleotide, peptide, peptidomimetic, monoclonal antibody or derivative thereof, including fragments and single chain antibodies.

### CROSS REFERENCE TO RELATED APPLICATIONS

This application claims the benefit of priority of U.S. Provisional Application No. 60/238,942, filed Oct. 10, 2000.

### FIELD OF THE INVENTION

The invention relates to a conjugate of the expression protein of the Bcl-2 family, including Bax, Bak or Bak-2 genes and a ligand targeting a cell surface antigen of a specific cell type.

### BACKGROUND OF THE INVENTION

Apoptosis is a normal physiologic process that leads to individual cell death. This process of programmed cell death is involved in a variety of normal and pathogenic biological events and can be induced by a number of unrelated stimuli. Changes in the biological regulation of apoptosis also occur during aging and are responsible for many of the conditions and diseases related to aging. Recent studies of apoptosis have implied that a common metabolic pathway leading to cell death may be initiated by a wide variety of signals, including hormones, serum growth factor deprivation, chemotherapeutic agents, ionizing radiation and infection by human immunodeficiency virus (HIV). Wyllie (1980) Nature, 284:555-556; Kanter et al. (1984) Biochem. Biophys. Res. Commun. 118:392-399; Duke and Cohen (1986) Lymphokine Res. 5:289-299; Tomei et al. (1988) Biochem. Biophys. Res. Commun. 155:324-331; Kruman et al. (1991) J. Cell. Physiol. 148:267-273; Ameisen and Capron (1991) Immunology Today 12:102; and Sheppard and Ascher (1992) J. AIDS 5:143.

Apoptosis involves two primary steps. The Bcl-2 family of proteins are important in the “decision” step of apoptosis. In contrast, the “execution” phase of apoptosis is mediated by the activation of caspases, cysteine proteases homologous to the C. elegans protease ced-3, which induce cell death via the proteolytic cleavage of substrates vital for cellular homeostasis. Bcl-2-related proteins act upstream from caspases in the cell death pathway and recent studies demonstrated that another C. elegans gene, ced-4, or its mammalian homolog Apaf-1 can bridge between Bcl-2/ced-9 family members and caspases. Apoptotic cell death is characterized by cellular shrinkage, chromatin condensation, cytoplasmic blebbing, increased membrane permeability and interchromosomal DNA cleavage or DNA fragmentation. Kerr et al. (1992) FASEB J. 6:2450; and Cohen and Duke (1992) Ann. Rev. Immunol. 10:267. The blebs, small, membrane-encapsulated spheres that pinch off of the surface of apoptotic cells, may continue to produce superoxide radicals which damage surrounding cell tissue and may be involved in inflammatory processes.

Bcl-2 was discovered at the common chromosomal translocation site t(14:18) in follicular lymphomas and results in aberrant over-expression of bcl-2. Tsujimoto et al. (1984) Science 226:1097-1099; and Cleary et al. (1986) Cell 47:19-28. The normal function of bcI-2 is the prevention of apoptosis; unregulated expression of bcl-2 in B cells is thought to lead to increased numbers of proliferating B cells, which may be a critical factor in the development of lymphoma. McDonnell and Korsmeyer (1991) Nature 349:254-256; and, for review see, Edgington (1993) Bio/Tech. 11:787-792. Bcl-2 is also capable of blocking gamma irradiation-induced cell death. Sentman et al. (1991) Cell 67:879-888; and Strassen (1991) Cell 67:889-899. It is now known that bcl-2 inhibits most types of apoptotic cell death and is thought to function by regulating an antioxidant pathway at sites of free radical generation. Hockenbery et al. (1993) Cell 75:241-251. While apoptosis is a normal cellular event, it can also be induced by pathological conditions and a variety of injuries, including, e.g., cardiovascular diseases such as ischemia/reperfusion injury, cancer regression, immunoregulation, viral diseases, anemia, neurodegenerative disorders, gastrointestinal disorders, such as diarrhea and dysentery, diabetes, hair loss, rejection of organ transplants, prostate hypertrophy, obesity, ocular disorders, stress, and aging.

Bcl-2 belongs to a family of proteins some of which have been cloned and sequenced. Williams and Smith (1993) Cell 74:777-779. Prominent among these is the BCL-2 family of proteins whose members include dominant suppressors of cell death (Ced-9, BCL-2, BCL-XL, BCL-w, A1, and MCL-1) and proapoptotic inducers of cell death (BAX, BAK, and BCL-Xs), as well as proapoptotic inhibitors of BCL-2/BCL-XL function (BAD, BID) (Yang and Korsmeyer, Blood 88:386-401 (0996)). The proapoptotic inducers Bax, Bak and Bak-2 have been described (see, e.g., U.S. Pat. Nos. 5,998,131; 6,015,687).

Apoptosis is important clinically for several reasons. In the field of oncology, many of the clinically useful drugs kill tumor cells by inducing apoptosis. For example, cancer chemotherapeutic agents such as cisplatin, etoposide and taxol all induce apoptosis in target cells. In addition, a variety of pathological disease states can result from the failure of cells to undergo proper regulated apoptosis. The development of efficacious compounds which are capable of specifically inducing apoptosis would therefore be of therapeutic value in the treatment of these pathological diseases states.

### DESCRIPTION OF THE INVENTION

The invention relates to a conjugate of a pro-apoptotic protein, e.g., the expression protein of the Bak, Bak-2 or Bax genes, and a ligand targeting a cell surface antigen of a specific cell type. The ligand can be any molecule capable of targeting a cell surface antigen, including organic molecules, for example, oligonucleotides, peptides, peptidomimetics, monoclonal antibodies and derivatives thereof, including fragments and single chain antibodies. These conjugates of the invention can be used for eliminating cells through apoptosis. This allows depletion or elimination of tumor cells, infected cells, mast cells or basophils, or autoreactive cells, and makes the conjugates useful in treating, respectively, tumors, cancers, infectious diseases (including viral diseases), allergic diseases and autoimmune diseases.

The cell surface antigen may be expressed, e.g., on particular types of cells, or narrow classes of cells, to prevent wide-ranging cellular apoptosis from resulting. Apoptosis in broad classes of cells could cause serious adverse effects, including, for example, immunosuppression if T cells or other immune system cells are depleted.

If antibodies are used as the targeting moiety in the conjugate, for in vivo use the antibodies would preferably be chimeric, Delmmunised™, humanized or human antibodies. Such antibodies can reduce immunogenicity and thus avoid human anti-mouse antibody (HAMA) response. The antibody can be IgG1 or IgG3 to augment antibody-dependent cellular cytotoxicity (S. M. Canfield et al., J. Exp. Med., 1991; 173: 1483-1491) and complement mediated cytolysis (Y.Xu et al., J. Biol Chem., 0994; 269: 3468-3474; V. L. Pulito et al., J. Immunol., 1996; 156: 2840-2850), and aid in elimination of the targeted cells.

Chimeric antibodies are produced by recombinant processes well known in the art, and have an animal variable region and a human constant region. Humanized antibodies have a greater degree of human peptide sequences than do chimeric antibodies. In a humanized antibody, only the complementarity determining regions (CDRs) which are responsible for antigen binding and specificity are animal derived and have an amino acid sequence corresponding to the animal antibody, and substantially all of the remaining portions of the molecule (except, in some cases, small portions of the framework regions within the variable region) are human derived and correspond in amino acid sequence to a human antibody. See L. Riechmann et al., Nature, 1988; 332: 323-327; G. Winter, U.S. Pat. No. 5,225,539; C. Queen et al., U.S. Pat. No. 5,530,101.

Delmmunised™ antibodies are antibodies in which the T helper epitopes have been eliminated, as described in International Patent Application PCT/GB98/01473. They have either no immunogenicity or reduced immunogenicity when applied in vivo.

Human antibodies can be made by several different ways, including by use of human immunoglobulin expression libraries (Stratagene Corp., La Jolla, Calif.) to produce fragments of human antibodies (VH, VL, Fv, Fd, Fab, or F(ab′)₂) and using these fragments to construct whole human antibodies using techniques similar to those for producing chimeric antibodies. Human antibodies can also be produced in transgenic mice with a human immunoglobulin genome. Such mice are available from Abgenix, Inc., Fremont, Calif., and Medarex, Inc., Annandale, N.J.

One can also create single peptide chain binding molecules in which the heavy and light chain Fv regions are connected. Single chain antibodies (“scFv”) and the method of their construction are described in U.S. Pat. No. 4,946,778. Alternatively, Fab can be constructed and expressed by similar means (M. J. Evans et al., J. Immunol Meth., 1995; 184: 123-138). All of the wholly and partially human antibodies are less immunogenic than wholly murine MAbs, and the fragments and single chain antibodies are also less immunogenic. All these types of antibodies are therefore less likely to evoke an immune or allergic response. Consequently, they are better suited for in vivo administration in humans than wholly animal antibodies, especially when repeated or long-term administration is necessary. In addition, the smaller size of the antibody fragment may help improve tissue bioavailability, which may enhance dose accumulation of the conjugate.

Techniques useful for the practice of the present invention are described in a variety of references, including but not limited to, Harlow et al. (1988) “Antibodies: a Laboratory Manual,” Cold Spring Harbor Press, Cold Spring Harbor, N.Y.; Molecular Cloning: A Laboratory Manual, 2nd ed., Vol. 1-3, eds. Sambrook et al. Cold Spring Harbor Laboratory Press (1989); and Current Protocols in Molecular Biology, eds. Ausubel et al., Greene Publishing and Wiley-lnterscience: New York (1987) and periodic updates.

Based on the molecular structures of the variable regions of suitable targeting antibodies, one could use molecular modeling and rational molecular design to generate and screen small molecules that mimic the molecular structures of the binding region of the antibodies. These small molecules can be peptides, peptidomimetics, oligonucleotides, or other organic compounds. The mimicking molecules can be used as the targeting moeity in the conjugate. Alternatively, one could use large-scale screening procedures commonly used in the field to isolate suitable small molecules form libraries of combinatorial compounds.

The conjugates of this invention can be administered to patients in an appropriate pharmaceutical formulation by any route, which allows them to access the target antigens. Such routes include intravenous infusion, intravenous bolus injection, and intraperitoneal, intradermal, intramuscular, subcutaneous, intranasal, intratracheal, intraspinal, intracranial, and oral routes.

What is claimed is:

1. A conjugate comprising a pro-apoptotic protein and a ligand specifically targeting a cell surface antigen, wherein the pro-apoptotic protein is the expression protein of the Bak, Bak-2 or Bax genes.

2. The conjugate of claim 1 wherein the cell surface antigen is expressed on tumor cells, cells infected with bacteria or virus, or cells of specific functions, but it is not expressed or expressed only to a limited extent on other cell types.

3. The conjugate of claim 1 or 2 wherein the ligand is an organic molecule, an oligonucleotide, peptide, peptidomimetic, monoclonal antibody or derivative thereof, including fragments and single chain antibodies.

4. The conjugate of claim 3 wherein the monoclonal antibody is a chimeric, Delmmunised™, humanized or human antibody, and the fragment is Fv, Fd, Fab, or F(ab′)₂.
